(19) United States
(12) Patent Application Publication
(10) Pub. No.: US 2002/0198597 A1
(43) Pub. Date: Dec. 26, 2002

(54) Prosthetic eyelashes

(76) Inventor: Yvonne Godfrey, Lake Ronkonkoma, NY (US)

Correspondence Address: BRYAN CAVE LLP, 245 Park Avenue, New York, NY, 10167, US

(21) Appl. No.: 10/124,866

(22) Filed: Apr. 18, 2002

Related U.S. Application Data
(60) Provisional application No. 60/284,631, filed on Apr. 18, 2001.

(51) Int. Cl. A61F 2/10

(52) U.S. Cl. 623/15.11

(57) ABSTRACT

A device for eyelash replacement is described having a band portion, having at least one layer front outer layer of material simulating human skin on a first side of the band, the band having a second side opposite from the first said, the second side being removeably adherable to at least a portion of a user's upper or lower eyelid. A plurality of follicles is coupled to the layer of the band and the band is affixed to a user by adhering the band portion second side to a user's eyelid. A method is also described for affixing a hair follicle having a near end and a distal end to a band having at least one layer of simulated human skin. The method involves perforating the layer of simulated human skin so as to create an aperture having a pre-determined diameter, inserting the follicle near end through the aperture such that a length of follicle is under the layer, and, knot-tying the length of the follicle near end into a knot of a size diametrically larger than the diameter of the aperture.

### PRIORITY

This application claims the benefit of U.S. Provisional Application No. 60/284,631 filed Apr. 18, 2001.

### FIELD OF THE INVENTION

The invention is directed to the field of cosmetic hair and skin replacement, and more particularly to the field of replacement eyelashes.

### BACKGROUND OF THE INVENTION

Use of “false eyelashes” as a cosmetic accessory has a long history. Ordinarily, however, false eyelash systems and devices are almost exclusively made for people who want to enhance existing lashes, i.e. make them thicker, more colorful or longer. Such may be in a unitary piece, supported on the wearer's existing lashes or may also include application of single eyelashes to a wearer's existing lashes to provide a fuller appearance.

To date, the cosmetic market has largely ignored those potential customers who are seeking an eyelash system to replace eyelashes lost, for instance through chemotherapy, burns or other skin conditions. Surgical solutions for such persons exist, but involve considerable expense and discomfort.

Those seeking to totally replace eyelashes find only a narrow field of potential replacements that are convenient and yet provide effective and pleasing results. Most eyelash replacement systems rely on existing lashes to either blend or support the artificial lashes. The emphasis is more on supplementing existing lashes over substitution for lashes lost.

For those without eyelashes, using existing systems are, at best, a challenge. To date, replacement eyelashes were simply not meant to replace all lashes a person may have lost.

### SUMMARY OF THE INVENTION

A device for eyelash replacement is disclosed comprising a band portion, having at least one layer of material simulating human skin on a first side of the band, the band having a second side opposite from the first said, the second side being removeably adherable to at least a portion of a user's upper or lower eyelid, a plurality of follicles coupled to the at least one layer of the band, and, a means for adhering the band portion second side to a user's eyelid.

A method is also disclosed for affixing a hair follicle having a near end and a distal end to a band having at least one layer of simulated human skin comprising perforating the layer of simulated human skin so as to create an aperture having a pre-determined diameter, inserting the follicle near end through the aperture such that a length of follicle is under the layer, and knot-tying the length of the follicle near end into a knot of a size diametrically larger than the diameter of the aperture.

### DETAILED DESCRIPTION OF THE INVENTION

The eyelash replacement of the instant invention uses human hair follicles cut to appropriate “natural” length for human eyelashes. The lashes need not only be made of human hair, other natural and synthetic alternatives exist which could be used instead. Filaments made of polymer fibers, for instance, could be employed to impart strength over natural hair.

Typically, such lashes, whether natural or synthetic, would be about approximate to the length and thickness of human eyelash hairs, preferably having a mix of varying lengths to provide an even distribution when these are applied to the carrying device, which in the preferred embodiment is a thin band made of flexible, human skin-like material. Usually a mixture of follicles cut to longer or shorter lengths provides a sufficient blend giving the look and feel of even distribution of lengths and thickness.

The follicles are affixed to the band along the band length. Standard lengths for the band are approximately 2.8 cm long, with a width approximately 4 mm. Other band sizes for eyelash replacement over less than the fall length of an eyelid, or for children or other smaller sized eyelids are also within the scope of the invention. Starting from one end of the band, smaller lengths may be used with the length and thickness of the lashes becoming increasingly longer as the lashes are applied along the band. The band may be arcuate or semi-circumferential in shape to follow a general eyelid contour, or it may be made of flexible material which is freely bendable to fit any arcuate angle. Preferably, some arcuate structure is used to provide stability to assist grasping the band for affixation. Other lengths and widths for the band, depending on the surface area to be covered, for instance, a portion of the user's eyelid if the user only has partial eyelash loss in one region, are within the scope and contemplation of the invention.

The Band is preferably of a flexible material which simulates natural human skin in both color and texture. Polymer based materials of a particular texture and flexibility are preferred. In the alternative, the band may be made of soft, yet rigid material, cut in the shape desired of the eyelid to a pre-determined arcuate angle to fit the eyelid of a user. The instant invention may be used to replace eyelashes on both the upper or lower eyelids for a given eye. The “lower” eyelid may be defined as the region formed of skin which meets with the upper eyelid when the eye is closed.

The Follicles may be placed or shaped within the band to reproduce or enhance the eyelid region for which the replacement lashes are sought. If the less dense lower eyelid eyelashes are to be replaced, the spacing of the follicles may be further apart or the patterns altered accordingly to suit a natural appearance of eyelash distribution. Consequently, more dense placement of follicles on the upper eyelash may either reproduce the former eyelashes, now lost, or provide a cosmetic enhancement or improvement over the former lashes. The follicles may be curled or shaped to a pre-determined degree to reproduce the curvature of natural eyelashes. Some curling of the hair or fiber used may be necessary prior to affixation to the band to provide a minimum amount of “natural” curl. The hair or synthetic material used may also be sufficiently flexible and pliable to allow the user to shape the follicles using conventional eyelash curling devices.

The band is composed of layered material simulating the layered quality of human skin, i.e. composed of various accumulated layers of skin. The band has multiple layers of flexible, rigid or semi-rigid material. Each layer has a pre-determined thickness in the approximate thickness of a layer of human skin, or, in any event, between 10 and 150 μm, and, in particular, under about 50 μm such that the accumulation of layers does not appreciably raise-up or protrude when the band portion with affixed lashes as a whole is applied. The specific number of layers is not critical so long as there sufficient layers to provide structural stability for the insertion of the eyelashes, ordinarily this can be accomplished with at least two layers, one outer and one inner layer.

The types of materials usable are, for instance, polymer based sealer type derivatives which provide a clear, transparent, yet soft and flexible molded material to form the band. These derivatives may be in a liquid or gel type solution that may be spread in thin accumulating layers, approximate to the thickness of human skin. These polymer based sealer type derivatives may be polymeric composites which simulate natural human skin in both appearance and texture and may be produced, for instance, from composites of polyurethane resin which have a low hardness or become soft and flexible by addition of a plasticizers or fillers to enhance softness and/or flexibility, e.g., up to about 10% silicon filler, which may be in the form of silicon oxide. Commercial brands of suitable materials include BIO-SKIN from Advanced Hair Genesis of Boca Raton, Fla., or SKIN REPLICA from New Hair Concepts of Pompano Beach, Fla., or VIRTUAL SKIN from American Hairlines of Sunrise, Fla. These composites may be prepared in thin layers which are aggregated and then molded into the band by molding methods known in the art.

Preferably the band material composition, including any flexibility imparted by a filler such as silicon oxide compounds combined with the transparency and rigidity of the polyurethane resin, render the Band material nearly indistinguishable from the visible skin of the user's eyelid upon application to the user's upper or lower eyelid.

The skin-like material of the band includes at least one outer layer at the front side of the band where eyelash follicles extend out from, and one outer layer at the rear side of the band. The band front outer layer includes apertures through which the follicles extend. These apertures are created by piercing through the band front outer layer when the follicles are inserted.

The eyelash system may be constructed using a precision sewing process. The follicles can be inserted by hook and loop injection conducted by, for instance, industrial precision sewing apparatuses operating over a cutting bed which inject the hair into the band and tighten the loop to form a knot larger than the size of the aperture, or by other means known in the art. Once inserted, the follicles may be micro-knotted to form a knot at the inserted end with a diameter larger than the diameter of the apertures through which the follicles are inserted. Thus, the follicles are retained between the outer layers and other intervening layers which may also be used to stabilize and hold them in place. Excess lengths of follicles are then trimmed.

Alternatively, the follicles may applied in a layering process during fabrication of the band and be placed over at least one layer of skin material after it is spread, for instance, in a mold. Later layers of skin material may then be applied over the follicles so placed and dried to retain the follicles between at least two layers of the skin material. Excess lengths of follicles are then trimmed.

It is also within the scope and contemplation of the invention that the sewing process for inserting the follicles into the band be combined with the layering process, for instance, the precision insertion of follicles through at least one layer of the skin material of the band, then a further layer of skin material superimposed, either by a thin liquid layer or by heat fusion of a sheet or aggregate multiple layers, over the point where the precision sewing stitching occurred on the band.

The preferred manner of affixation of the eyelash band to a user's eyelid is through application of an adhesive. In one embodiment, the band rear outer layer may be treated with an adhesive to apply the band to a wearer's eyelid. The adhesive used may be a water-proof rubber latex adhesive, preferably applied in a thin layer to either the band or the eyelid where the band will be affixed.

While the invention has been described in respect to the above embodiments of the invention, it should be understood that the invention is not limited to these precise embodiments. Rather, many modifications and variations will present themselves to persons skilled in the art without departure from the scope and spirit of the inventions, which is defined in the appended claims.

### BRIEF DESCRIPTION OF THE DRAWINGS

FIG. 1 illustrates a front view of the eyelash device of one embodiment of the invention.

FIG. 2 illustrates a frontal view of the eyelash device of the embodiment of FIG. 1 in which the transparent nature of the band material is visible.

FIG. 2a illustrates a cutaway side cross-section of the eylash device of FIG. 2.

FIG. 3 illustrates a manner of affixing the eyelash device to an eyelid of a user.

### DETAILED DESCRIPTION OF THE DRAWINGS

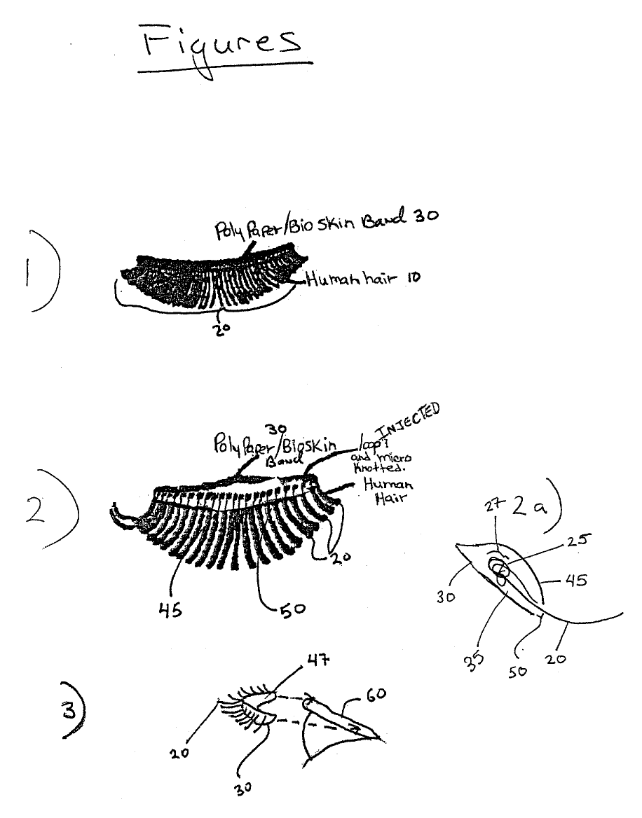

FIG. 1 shows a frontal view of the eyelash replacement device of one embodiment of the invention. Human hair 10 consists of individual follicles 20 cut to the appropriate length to simulate “natural” eyelashes. Follicles 20 are affixed to band 30, where band 30 is preferably of a flexible material which simulates natural human skin in texture. Band 30 is preferably of an arcuate shape, or flexible to conform to an arcuate shape, to be affixed to the eyelid of a wearer. Follicles 20 may be curved or brushed to assume a pleasing shape akin to the curl of natural eyelashes.

FIG. 2 illustrates a front view of the eyelash replacement device of FIG. 1 emphasizing transparent band 30. Band 30 may be made of a transparent material such that when affixed to a wearer's eyelid, the band material does not obstruct the appearance of the wearer's eyelid skin through the band 30. Thus, the band 30 becomes difficult to detect while affixed to a wearer and blends into the wearer's natural skin. Band 30 is composed of layers of skin-like material and has a front outer layer 45 covering over follicles 20 near end 27. Apertures 50 have a pre-determined diameter, and are created by piercing band 30 front outer layer 45 and inserting follicles 20 by using a precision sewing instrument. Follicles 20, at their near end 27, are knotted 25, to a knot diameter size larger than the diameter size of apertures 50, so follicles 20 cannot slip out of band 30.

FIG. 2a provides a detail cutaway view of FIG. 2 wherein follicle 20 is disposed in space 35, formed by outer layer 45 and inner layer 55 of band 30. Knot 25 is created from a loop of hair at follicle 20 near end 27 that is pulled into a knot during insertion of follicle 20 into band 30 through aperture 50 pierced through outer layer 45 by a precision sewing instrument.

FIG. 3 illustrates a manner of affixation of the eyelash band to a user's eyelid. Band 30 includes rear layer 47 opposing outer layer 45 and providing a surface which may be treated with an adhesive (not pictured) to apply band 30, with follicles 20 distal end 29 protruding from the opposing outer layer 45 away from the user's eyelid 60. When affixed with adhesive to the wearer's eyelid 60, band 30 made of transparent material, does not obstruct a view of the eyelid 60 covered by the band 30, thus blending in with the wearer's skin to avoid detection.

What is claimed is:

1) A device for eyelash replacement comprising:
a band portion, having at least one layer of material simulating human skin on a first side of the band, the band having a second side opposite from the first said, the second side being removeably adherable to at least a portion of a user's upper or lower eyelid;
a plurality of follicles coupled to the at least one layer of the band;
means for adhering the band portion second side to a user's eyelid.

2) The device of claim 1, wherein, the band portion has a plurality of layers of material simulating human skin.

3) The device of claim 1, wherein the band portion material simulating human skin is a thin layer of polymeric material comprising silicon and polyurethane resin.

4) The device of claim 2, wherein the follicles are coupled to the band portion between a first layer and a second layer of the plurality of band layers.

5) The device of claim 1, wherein the portion of a user's eyelid has an arcuate shape and the band portion is a flexible polymer based composite which conforms to the shape.

6) The device of claim 1, wherein the portion of a user's upper or lower eyelid has a shape and the affixation means is a liquid based polymer composite which adheres to the portion of the user's upper or lower eyelid.

7) The device of claim 1, wherein the means for adhering is a latex adhesive applied to the portion of the user's upper or lower eyelid.

8) A prosthetic eyelash replacement comprising:
a multi-layered band portion of synthetic skin material having a front outer layer and a rear outer layer;
an adhesive material applied to the rear outer layer of the band portion for affixation to a portion of a user's upper or lower eyelid;
a plurality of natural human hair follicles affixed to the band portion so as to be disposed between the front outer layer and the rear outer layer through a plurality of apertures in the first layer, the plurality of follicles each having a knot with a diameter larger than the diameter of the aperture such that the aperture blocks movement of the follicle therethrough.

9) The prosthetic eyelash replacement of claim 8, wherein the band portion synthetic skin material is a polymeric material comprising silicon and polyurethane resin.

10) The prosthetic eyelash replacement of claim 9, wherein the band portion skin material is translucent so as to allow visibility of a wearer's underlying skin when affixed to the wearer's eyelid.

11) The prosthetic eyelash replacement of claim 10, wherein the band portion skin material layers have a predetermined thickness approximately the thickness of a single layer of human skin.

12) A method of affixing a hair follicle having a near end and a distal end to a band having at least one layer of simulated human skin comprising:
perforating the layer of simulated human skin so as to create an aperture having a pre-determined diameter;
inserting the follicle near end through the aperture such that a looped length of follicle is under the layer such that the follicle distal end protrudes from the layer;
knot-tying the looped length of the follicle near end into a knot, the knot having a diameter of a size larger than the diameter of the aperture;

13) The method of claim 12 further comprising superimposing a further layer of simulated human skin on the band over the follicle near end.

14) The method of claim 12 further comprising molding the band from a silicon and polyurethane composite.

15) The method of claim 12 further comprising cutting the follicle distal end to a predetermined length.
